(19) United States
(12) Patent Application Publication
(10) Pub. No.: US 2026/0014321 A1
(43) Pub. Date: Jan. 15, 2026

(54) Ergonomic Syringe Attachment

(71) Applicant: Rachel Steward, M.D., A Medical Corporation, Los Angeles, CA (US)

(72) Inventors: Rachel Steward, Los Angeles, CA (US); Aaron Ryczek, Eastvale, CA (US)

(73) Assignee: Rachel Steward, M.D., A Medical Corporation, Los Angeles, CA (US)

(21) Appl. No.: 18/767,408

(22) Filed: Jul. 9, 2024

(51) Int. Cl. A61M 5/31

(52) U.S. Cl. CPC A61M 5/3137; A61M 2205/586

(57) ABSTRACT

An ergonomic syringe attachment may mitigate the risks of repetitive motion injuries including musculoskeletal conditions, pain, and loss of motion. These designs may allow a user to depress a syringe without relying principally on thumb pressure. An ergonomic syringe attachment may include a body coupled to a handle, and a base plate. The base plate may include claws which may form a channel configured to accept a shaft of the syringe. A thumb rest of the syringe may be held between the body and the base plate of the ergonomic syringe attachment. The ergonomic syringe attachment may enable a user to comfortably push fluids from or draw fluids into the syringe using the force of their bicep.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### TECHNICAL FIELD

This disclosure relates generally to the field of syringe accessories. More particularly, the present disclosure relates to an ergonomic syringe attachment.

### DESCRIPTION OF RELATED TECHNOLOGY

The stress from repetitive motion and the overuse of certain body parts can cause various musculoskeletal conditions. Tendinitis, osteoarthritis, and other repetitive strain injuries can result from such stress. Workers in hospitals and clinical settings, such as nurses, have an increased risk of musculoskeletal conditions due to performing repetitive tasks such as administering injections which can cause arthritis of the thumb and tendinitis of the wrist. These conditions can cause tenderness and pain and limit the strength and range of motion of sufferers.

To mitigate such risks, nurses and other healthcare workers use different parts of their body to depress the syringe (e.g., their belly, thigh, or other hand). Stretching may also be used to combat the effects of the stress of repetitive motion. Over the counter and prescription pain relievers may be used to combat the pain and inflammation. Surgery may be used to treat severe cases of arthritis.

### BRIEF DESCRIPTION OF THE DRAWINGS

FIGS. 1 and 2 are perspective views of an ergonomic syringe attachment with attached syringe according to aspects of the present disclosure.

FIG. 3 is an exploded view of the ergonomic syringe attachment of FIGS. 1 and 2.

FIG. 4 is a top view of the body of the ergonomic syringe attachment of FIGS. 1 and 2.

FIG. 5 is a cross-sectional view of the handle of the ergonomic syringe attachment of FIGS. 1 and 2.

FIG. 6 is an underside view of the base plate mount of the body of the ergonomic syringe attachment of FIGS. 1 and 2.

FIG. 7 is a side view of the body of the ergonomic syringe attachment of FIGS. 1 and 2.

FIGS. 8 and 9 are perspective views of an ergonomic syringe attachment according to aspects of the present disclosure.

### DETAILED DESCRIPTION

In the following detailed description, reference is made to the accompanying drawings which form a part hereof wherein like numerals designate like parts throughout, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense, and the scope of embodiments is defined by the appended claims and their equivalents.

Aspects of the disclosure are disclosed in the accompanying description. Alternate embodiments of the present disclosure and their equivalents may be devised without departing from the spirit or scope of the present disclosure. It should be noted that any discussion herein regarding “one embodiment”, “an embodiment”, “an exemplary embodiment”, and the like indicate that the embodiment described may include a particular feature, structure, or characteristic, and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

Example embodiments will now be described more fully with reference to the accompanying drawings. For purposes of the description hereinafter, it is to be understood that the embodiments described below may assume alternative variations and embodiments. It is also to be understood that the specific articles, compositions, and/or processes described herein are exemplary and should not be considered as limiting. In the description, relative terms such as “lower,” “upper,” “front,” “rear,” “horizontal,” “vertical,”, “above,” “below,” “up,” “down,” “top,” “bottom,” and “side” as well as derivatives thereof (e.g., “horizontally,” “downwardly,” “upwardly,” etc.) should be construed to refer to the orientation as then described or as shown in the drawing under discussion. The top of the ergonomic syringe attachment, for example, is the direction toward the handle from the base. The underside of the ergonomic syringe attachment or the underside of a component of the ergonomic syringe attachment, for example, is the area not facing the handle end of the ergonomic syringe attachment. The rear of the ergonomic syringe attachment or the rear of a component of the ergonomic syringe attachment, for example, is the direction opposite the opening/central channel of the ergonomic syringe attachment. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

Example Operation

According to aspects of the present disclosure, ergonomic syringe attachment designs are disclosed to mitigate the risks of musculoskeletal conditions. These designs may allow a user to depress a syringe without relying principally on thumb pressure. Instead, these ergonomic designs may allow the force of depressing a syringe to be supplied by a user's arm/bicep which may relieve pressure on the thumb of a user. The use of these ergonomic syringe attachment designs may allow a user to repetitively depress syringes with a lower long-term risk of musculoskeletal conditions and its associated pain and loss of motion.

Additionally, in some aspects, the disclosed designs may be small and portable which can be held in the pocket of a user (e.g., a healthcare worker) who might need regular access to the ergonomic syringe attachment in the performance of their job responsibilities. The disclosed designs may also work with a wide variety of syringe types and sizes. Loading syringes into and removing syringes from the disclosed designs may be simple and fast promoting their repeated use by healthcare workers. The disclosed designs may include no or few moving parts that may wear out, allowing for long term use throughout the career of a healthcare worker.

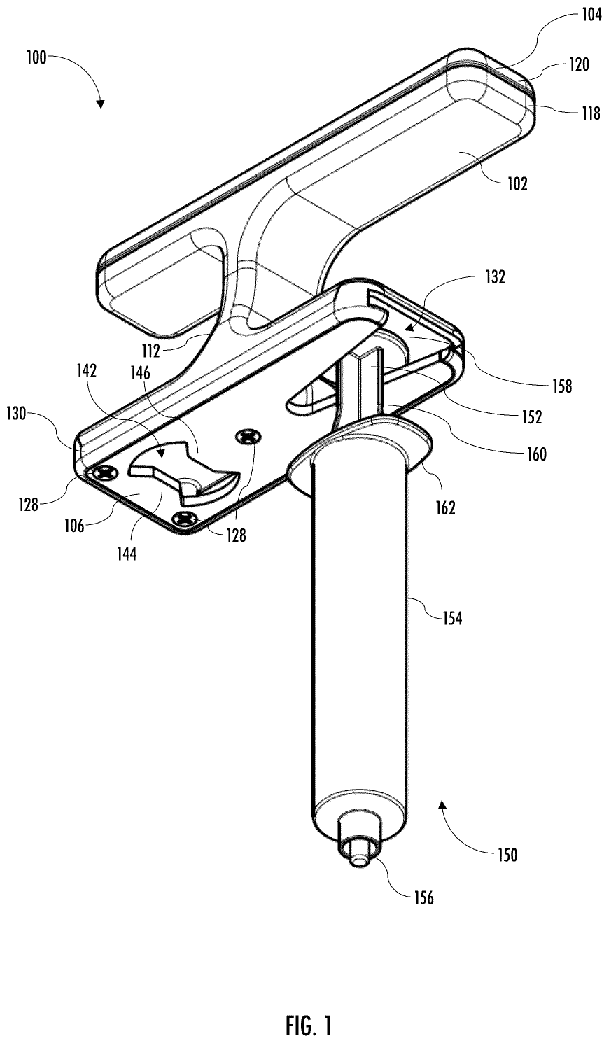

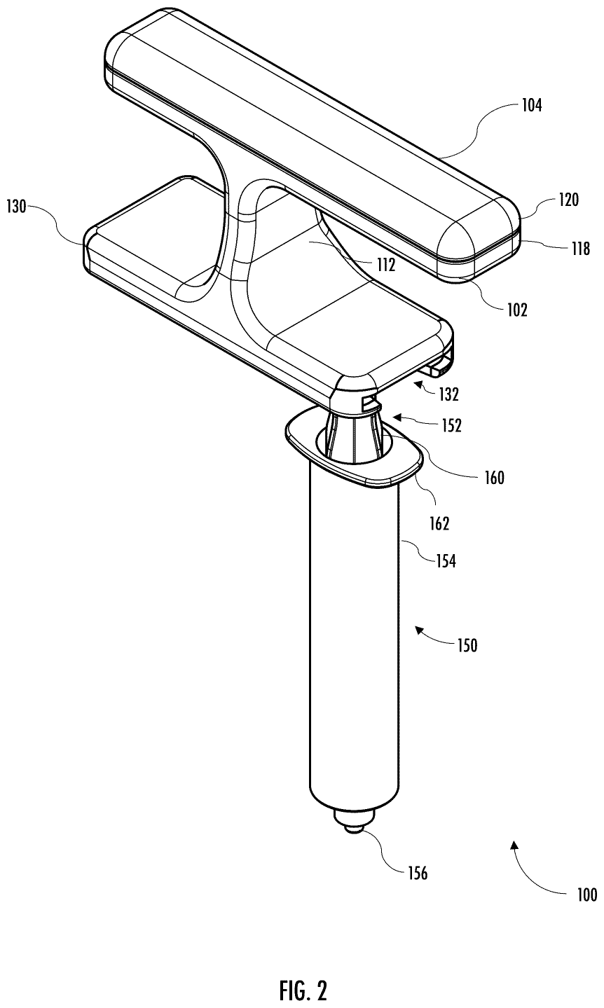

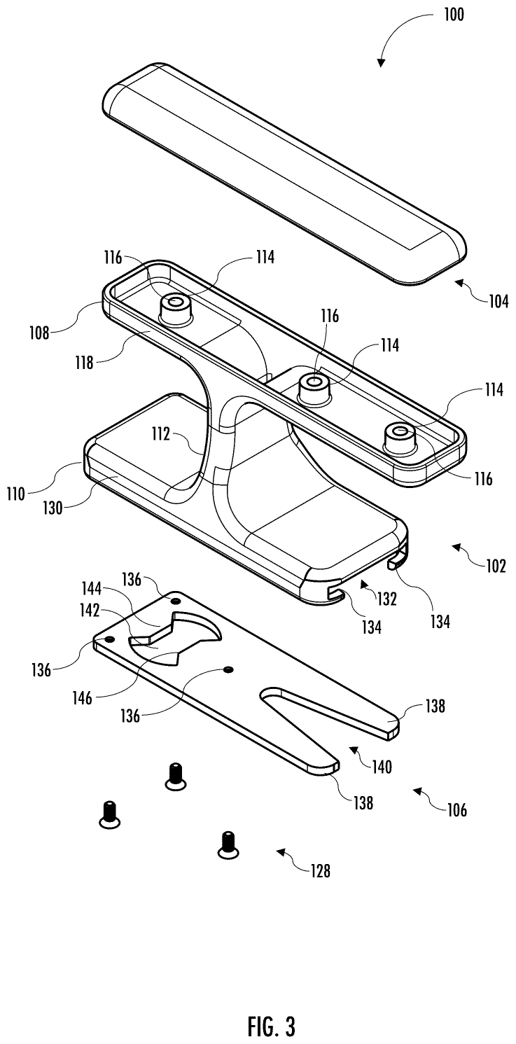

FIGS. 1 and 2 are perspective views of an ergonomic syringe attachment 100 with attached syringe 150 according to aspects of the present disclosure. FIG. 3 is an exploded view of the ergonomic syringe attachment 100. The ergonomic syringe attachment 100 may include a body 102 coupled to a handle 104, and a base plate 106. A syringe 150 may be held between the body 102 and the base plate 106 of the ergonomic syringe attachment 100. The ergonomic syringe attachment 100 may serve to elongate the syringe 150 and enable a user to comfortably push fluids from or draw fluids into the syringe 150 using the force of their bicep (rather than, for example, their thumb).

Syringes

As a brief aside, syringes are a type of reciprocating pump that includes a plunger/piston (e.g., plunger 152) that fits tightly within a barrel 154. The barrel 154 may be a cylindrical (or other shaped) hollow tube used for injecting or withdrawing liquids or gasses through a tip 156. Gradations on the sides of the barrel 154 may indicate the volume of fluid present in the barrel 154. Syringes are used in clinical medicine (to, e.g., inject medicine/vaccinations, withdraw blood) as well as in non-medical settings (e.g., in research laboratories, culinary, and industrial environments).

The tip 156 is an opening at one end of the barrel 154 opposite the opening for the plunger 152. The tip 156 may be opposite an opening in the barrel that accommodates the plunger 152. The tip 156 typically has a diameter smaller than the diameter of barrel 154. The tip 156 may be connected or connectable to various attachments including needles, intravenous (IV) tubing, and specialized tips (e.g., catheter tips, nozzles). The tip 156 may be outfitted with locks or screw threading to aid in attachment to form a tight-fitting/fluid-proof connection.

The plunger 152 includes a thumb rest 158 at one end connected to a shaft 160. The shaft 160 may be cylindrical or form another shape (e.g., a cross/X-shape as illustrated). The end of the shaft 160 opposite the thumb rest 158 may include a gasket. The gasket may be made of rubberized material and be configured to prevent fluid from leaking, or to create a vacuum inside the barrel 154, while the plunger 152 is pushed or pulled inside the barrel 154 of the syringe 150.

The barrel 154 may include a finger rest 162 to allow a user to hold and control the barrel 154 of the syringe 150 between their fingers (typically between the index and middle finger) to allow the user's thumb to depress the thumb rest 158 of the plunger 152.

Syringes 150 are produced in multiple sizes with barrels 154 of differing capacities from less than 1 mL to greater than 60 mL. The size and shapes of thumb rests 158 and shafts 160 may also vary between syringes 150. Healthcare workers may typically interact with syringes 150 of multiple sizes daily. Due to the repetitive motion of depressing the plunger 152 and given the varying types and sizes of syringe 150, aspects of the present disclosure provide an ergonomic syringe attachment 100 that can accommodate a wide variety of syringes 150 obviating the need of carrying attachments of varying sizes.

Ergonomic Syringe Attachment

The body 102 of the ergonomic syringe attachment 100 has a handle mount 108 and a base plate mount 110 joined by a neck 112. In some examples, the body 102 is constructed from a hard plastic (e.g., polycarbonate, polyethylene, thermoplastic resins such as acrylonitrile butadiene styrene (ABS), etc.) via injection molding, 3D-printing, etc. In other examples, the body 102 is constructed from a metal (e.g., aluminum, steel, titanium, etc.) via casting, 3D-printing, etc. Other durable and lightweight materials may be used to construct the body 102 with equal success. In some examples, the body 102 may have a hollow interior. This may allow the ergonomic syringe attachment 100 to reduce weight. In other examples, the body 102 may be substantially solid/filled. In a specific example, the body 102 has a height of 2.26 inches, however, those of ordinary skill will recognize that longer and shorter heights may be used with equal success.

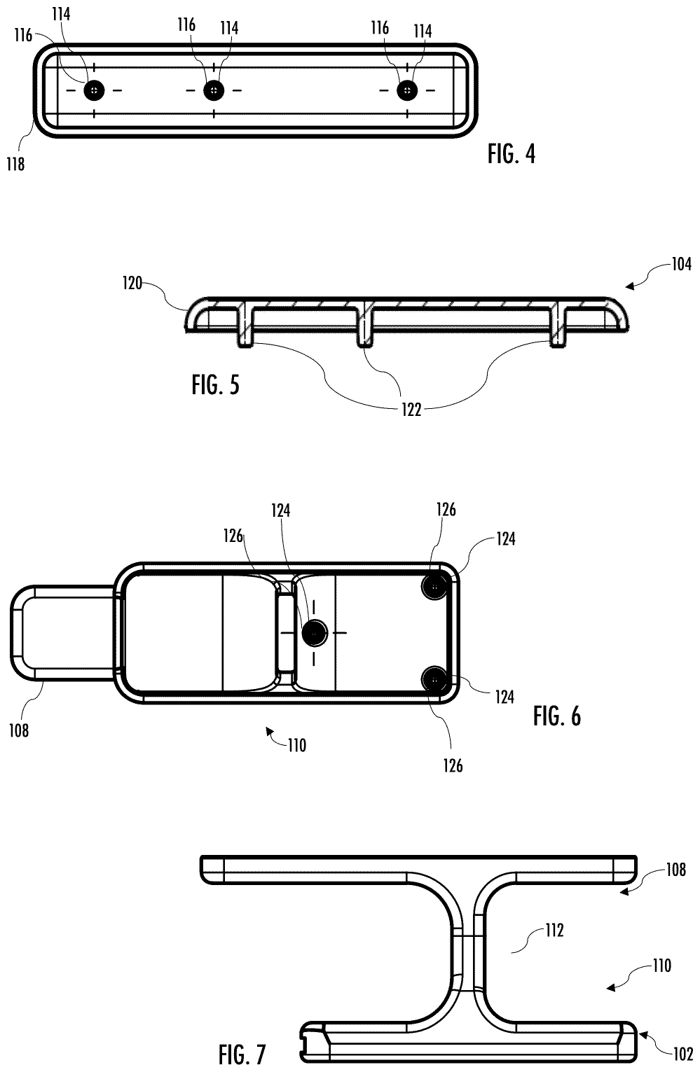

Referring also to FIG. 4, FIG. 4 is a top view of the body 102 of the ergonomic syringe attachment 100 according to aspects of the present disclosure. The handle mount 108 of the body 102 may be configured to connect to and support the handle 104. For example, bosses 114 or other protrusions with a cavity 116 may be used to mate with features on the handle 104. Sidewalls 118 of the handle mount 108 may reach the same height as the bosses 114 (e.g., 0.291 inches) from a base of the handle mount 018. As shown in FIG. 3, three bosses 114 may be used to couple the handle mount 108 to the handle 104. The bosses 114 may be reinforced with ribbing or a fillet to provide additional structural stability. In other examples, the handle mount 108 has a flush face that may be coupled to the handle 104. Adhesive glues or resins may be used to couple the handle 104 to the handle mount 108.

The handle 104 is configured to couple to the handle mount 108 of the body 102. The handle 104 may be rounded on the side portions of the outer wall 120 that meet the sidewalls 118 of the handle mount 108. The sidewalls 118 of the handle mount 108 may lay flush against the outer wall 120 of the handle 104. Joining of the handle 104 and the handle mount 108 may form a seam around the ergonomic syringe attachment 100 between the handle 104 and the handle mount 108 where the handle 104 and body 102 meet.

In some examples, the handle 104 is constructed from the same material as the body 102. In other examples, the handle 104 is constructed out of a tacky/grippy or soft touch plastic (e.g., a rubber, silicone, or rubber/low density plastic mix). The tacky/grippy or soft touch plastic may also be extruded or sprayed onto the handle 104 forming all or part of the outer wall 120. In some examples, the outer wall 120 is a smooth texture. In other examples, the outer wall 120 is textured (with, e.g., ridges, diamond plate pattern, a grit pattern, etc.) which may aid with gripping the handle 104. In additional examples, handle 104 includes padding (e.g., foam or with a covered filling) to aid in user comfort.

In some examples, the handle 104 (and/or handle mount 108) may have a larger (or smaller) width compared to the neck 112 or the base plate mount 110 to better fit various user hand sizes. For example, the handle mount 108 may have a first width (e.g., 1.00 inch) and the base plate mount 110 has a second width (e.g., 1.50 inches), which is larger than the first width of the handle mount 108. The neck 112 may have a width that thickens as it gets closer base plate mount 110 from the handle mount 108.

Referring also to FIG. 5, FIG. 5 is a cross-sectional view of the handle 104 of the ergonomic syringe attachment 100 according to aspects of the present disclosure. Handle 104 may have projections 122 on the interior side. The projections 122 may be configured to align with and fit within the bosses 114 of the handle mount 108. In other examples, the handle 104 has bosses and the handle mount 108 has projections configured to fit within the bosses. In some examples, the outer wall 120 may have a shallower height (e.g., 0.29 inches) compared to the projections 122 (e.g., 0.44 inches). In other examples, such as when the bosses 114 are longer than the sidewalls 118 of the handle mount 108, the outer wall 120 of the handle 104 may reach the same height as the projections 122 or the outer wall 120 may be shallower in height than the projections 122.

The base plate mount 110 may be configured to connect to and support the base plate 106. Referring also to FIG. 6, FIG. 6 is an underside view of the base plate mount 110 of the body 102 of the ergonomic syringe attachment 100 according to aspects of the present disclosure. On the underside of the base plate mount 110, three bosses 124 may be used to couple the base plate mount 110 of the body 102 to the base plate 106. For example, bosses 124 or other protrusions with a cavity 126 may be used to mate with features on the handle 104. In some examples, the bosses 124/cavity 126 is threaded (to accept attachment screws 128). In other examples, an internally threaded insert is affixed to each cavity 126 to accept attachment screws 128. Attachment screws 128 may be threaded to form a sturdy coupling between the base plate 106 and the base plate mount 110.

In an example, the bosses 124 are shallower than the sidewall 130 of the base plate mount 110 (e.g., the bosses 124 are 0.33 inches and the sidewall 130 is 0.43 inches). This may allow the base plate 106 (with a height of, e.g., 0.1 inches) to sit securely (e.g. inset) within the sidewall 130 and create a flush lower surface with the base plate mount 110. In other examples, the sidewall 130 of the base plate mount 110 may be the same height as the bosses 124 (e.g., 0.43 inches). The sidewall 130 of the base plate mount 110 may include an opening 132. The opening 132 may have a thickness configured to accept the thumb rest 158 of a syringe 150. The opening 132 may be a cutout in the sidewall 130 of the base plate mount 110. Cutouts in the sidewall 130 of the base plate mount 110 form projections 134. The cutouts and opening 132 allows the base plate mount 110 to accept the syringe 150. The projections 134 provide additional support of the base plate 106 from shifting laterally (e.g. through the opening 132).

The base plate 106 is configured to ensure the connection between the syringe 150 and the ergonomic syringe attachment 100 remains secure. The base plate 106 may be constructed from metal (e.g., stainless steel, aluminum, etc.), a durable plastic (e.g., polycarbonate), or any other material that can secure the syringe 150 onto the ergonomic syringe attachment 100. The base plate 106 may be a single sheet of uniform thickness (e.g., 0.1 inch). In other examples, portions of the base plate 106 are thicker than other areas (e.g., areas around the vial opener 142, V-shaped channel 140, etc.) to provide additional strength. The base plate 106 may secure the syringe 150 against lateral movement and/or rotation when engaged in the ergonomic syringe attachment 100. The base plate 106 may be secured to the base plate mount 110 with attachment screws 128 through attachment holes 136 in the base plate 106. The attachment holes 136 may be configured to align with bosses 124 of the base plate mount 110. In some examples, attachment holes 136 are spot-face or countersunk holes allowing the heads of the attachment screws 128 to sit flush against the surface of the base plate 106. Attachment screws 128 may include countersink screws having a flat head and a conical shape.

Claws 138 (or claw-like projections) may form a channel 140 configured to accept the shaft 160 of the syringe 150. In some examples, the claws 138 form a V-shaped channel 140 in the base plate 106. The claws 138 may form a curved (e.g., circular) end to the V-shaped channel 140. The claws 138 that form the V-shaped channel 140 are configured to latch onto the shaft 160 of the syringe 150 while also being able to accommodate multiple syringe sizes (e.g., 1 cc, 5 cc, 10 cc, 20 cc, etc.). In other examples, the claws 138 are parallel creating a rectangular channel 140 which may more securely fit a smaller range of shaft 160 sizes. In some examples, the base plate may have a length of 3.5 inches from an end to the claws 138 and a width of 1.3 inches. The claws 138 may have a length of 1.51 inches. The V-shaped channel 140 may have an opening of 19 degrees.

The base plate 106 may include a vial opener 142. Including a vial opener 142 on the ergonomic syringe attachment 100 may allow a user to replace a standalone bottle opener they may already carry. The vial opener 142 may include a cutout of the base plate 106. The vial opener 142 may include a lip 144 configured to catch the underside of a vial lid and a fulcrum 146 configured to receive the force that will remove the vial lid. The lip 144 and the fulcrum 146 may include trapezoidal (half hexagonal) projections within an oval/capsule shaped cutout. In some examples, the cutout area has a width of 99 inches, with a 0.44-inch gap between the lip 144 and the fulcrum 146. The trapezoidal projections of the lip 144 and fulcrum 146 may form a 97-degree angle. The vial opener 142 may be located between the attachment holes 136 in the base plate 106.

The neck 112 joins the handle mount 108 and the base plate mount 110 of the body 102 of the ergonomic syringe attachment 100. The neck 112 may be constructed from hard plastic or metal as the other components of the body 102. In other examples, the neck 112 is constructed separately from the handle mount 108 and the base plate mount 110 and later coupled using other materials. Other materials may be used to construct the neck 112 as would be appreciated by those of ordinary skill as long as the neck 112 provides sufficient strength and rigidity to transfer force from the handle 104/handle mount 108 to the base plate mount 110/base plate 106 and the plunger 152 of the syringe 150. The neck 112 may be configured so a user may hold the neck 112 between their fingers (e.g. between the index and middle or the middle and ring fingers) while grasping around the handle 104 and the handle mount 108 of the ergonomic syringe attachment 100 during operation. The neck 112 may extend across the entire widths of the handle mount 108 and/or the base plate mount 110. In other examples, the neck 112 may have a shorter width than the handle mount 108 and/or the base plate mount 110. In such examples, the neck 112 may be coupled at a central position along the widths of the handle mount 108 and/or the base plate mount 110. Referring also to FIG. 7, FIG. 7 is a side view of the body 102 of the ergonomic syringe attachment 100 according to aspects of the present disclosure.

The areas where the neck 112 joins the handle mount 108 and the base plate mount 110 may be curved. This may provide more comfort for a user (to curl their hands around the handle mount 108/handle 104) and aid in cleaning the ergonomic syringe attachment 100. In some examples, the neck 112 (or the area between the neck 112 and the handle mount 108) has indentions on one or each side configured to accommodate the placement of the sides of the user's fingers. The handle mount 108 may have finger indentions on the underside (away from the handle 104, toward the base plate mount 110) to aid user grip and add comfort during use.

In some examples, the handle mount 108 (and handle 104) is longer than the base plate mount 110 (and base plate 106). For example, the handle mount 108 may be 4.8 inches in length, whereas the base plate mount 110 is 3.7 inches. The neck 112 may couple to the handle mount 108 in an offset position from the center. The rear (vial opener 142) side of the base plate mount 110 and the rear side of the handle mount 108 may be aligned. The handle mount 108 may extend over the base plate mount 110 in the front. This may allow the force applied by the user to be directed more evenly to the plunger 152 of the syringe 150.

In other examples, the handle mount 108 (and handle 104) is the same length (or shorter) than the base plate mount 110 (and base plate 106). The neck 112 may join the handle mount 108 and the base plate mount 110 in the center. Alternatively, the neck 112 may be offset from the center of the handle mount 108 in an, e.g., uncentered position. In some examples, the front (opening 132) side of the base plate mount 110 may be aligned with the front side of the handle mount 108. This may also allow the force applied by the user to be directed more evenly to the plunger 152 of the syringe 150.

In some examples, the ergonomic syringe attachment 100 may be larger or smaller to better fit the hands of a user. For example, the neck 112 may be thinner (in smaller examples) or thicker (in larger examples) to provide a more custom fit. The lengths (and relative lengths) of the handle mount 108 and base plate mount 110) may vary between examples to accommodate different hand sizes.

Operation of the Ergonomic Syringe Attachment

A user may grasp the ergonomic syringe attachment (e.g., ergonomic syringe attachment 100) with their (dominant) hand. The user's palm may be placed on the handle and curve around the handle mount. The user may place their fingers around the handle mount with the neck of the ergonomic syringe attachment between the index and middle fingers (or middle and ring fingers) of the user.

Grasping a vial in their other (e.g., non-dominant) hand, the user may pry the cap off the vial by placing the lip of the vial opener on the underside of the cap of the vial and a top portion of the cap on the fulcrum of the vial opener and rotating the vial, the ergonomic syringe attachment, or both. This may dislodge the cap from the vial, exposing the contents of the vial.

The user may place the syringe into the ergonomic syringe attachment using their other (e.g. non-dominant) hand. The thumb rest of the syringe may be placed between the base plate mount and the base plate via the opening and between the claws of the base plate until the shaft of the plunger engages with the claws of the base plate.

The user may withdraw/draw medication from the vial into the syringe by holding the barrel of the syringe (and, in some examples, the vial) with their other (e.g., non-dominant) hand. Using force from their bicep, the user may pull the handle/handle mount of the ergonomic syringe attachment transferring the force onto the thumb rest of the syringe. This pulling motion may lift the plunger out of the barrel of the syringe withdrawing fluid from the vial. A user may use similar actions if withdrawing fluid from a tube connected to the syringe. After withdrawing fluid (e.g., medication or other fluid), tips/connections to e.g., tubing, may be removed/added from the syringe.

The user may push/administer fluid (e.g., medication) out of the syringe. The user may push/administer fluid from the syringe by holding the handle/handle mount of the ergonomic syringe attachment with one (e.g., their dominant) hand and the barrel of the syringe (and, in some examples, a receptacle/vial/tubing) with their other (e.g., non-dominant) hand. Using force from their bicep, the user may push the handle/handle mount of the ergonomic syringe attachment transferring the force onto the thumb rest of the syringe. This pushing motion may push the plunger (further) into the barrel of the syringe expressing fluid from the barrel of the syringe. The user may disconnect the syringe from any attachments.

The user may remove the syringe from the ergonomic syringe attachment by pulling the thumb rest/shaft of the syringe out from between the claws of the base plate and out the opening of the base plate mount.

U-Shaped Ergonomic Syringe Attachment

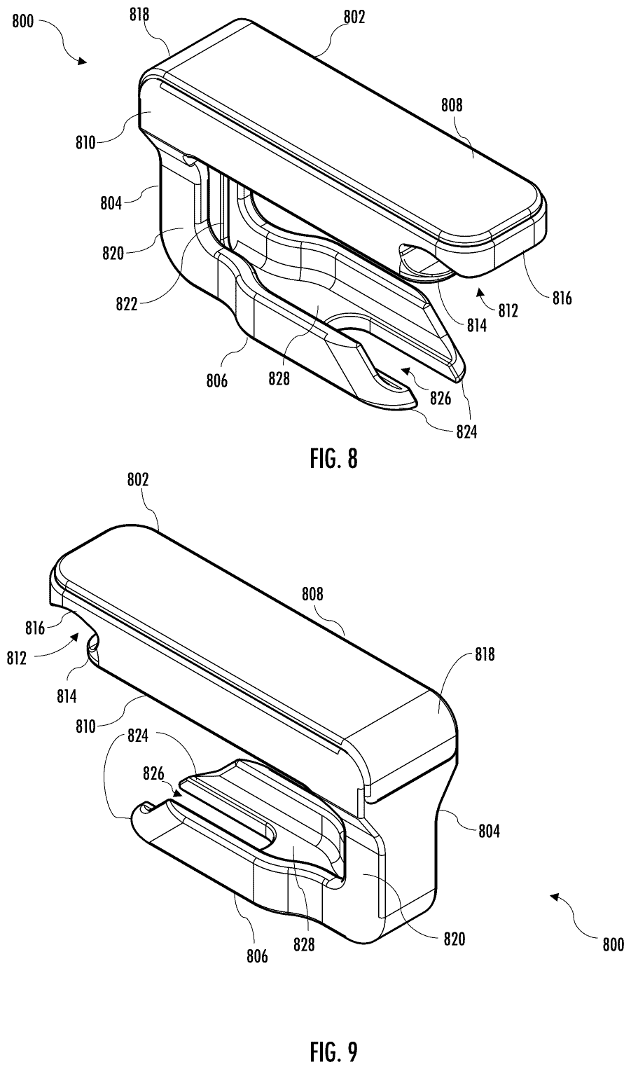

FIGS. 8 and 9 are perspective views of an ergonomic syringe attachment 800 according to aspects of the present disclosure. The ergonomic syringe attachment 800 may include a handle 802, a neck 804, and a base 806. The ergonomic syringe attachment 800 is removably couplable to a syringe (e.g., syringe 150 of FIGS. 1 and 2) at the base 806. The ergonomic syringe attachment 100 may be configured to enable a user to comfortably push fluids from or draw fluids into the syringe 150 using the force of their bicep (rather than, for example, their thumb). As shown, the design of the ergonomic syringe attachment 800 differs from the ergonomic syringe attachment 100 of FIGS. 1-7 in a number of ways to illustrate additional examples of the ergonomic syringe attachment 800 contemplated herein.

In some examples, the ergonomic syringe attachment 800 is constructed as a single piece constructed via, e.g., injection molding. In some examples, the single piece may be produced in multiple stages, for example via overmolding, to have different parts constructed from differing materials. This may obviate a need to use separate attachment mechanisms, e.g., attachment screws 128.

The handle 802 may include an upper portion 808 and a mount portion 810. The upper portion 808 and the mount portion 810 may be constructed out of two different types of molded plastics. For example, the upper portion 808 may be constructed from a thermoplastic rubber (TPR) configured to provide a soft grippable surface for the palm of a user. The mount portion 810 of the handle 802 (and the rest of the ergonomic syringe attachment 800) may be constructed from a durable thermoplastic, e.g., Acrylonitrile Butadiene Styrene (ABS), or other durable plastic.

The handle 802 may include a finger grip 812. The finger grip 812 may be located on the opposite end of the handle from the neck 804. The finger grip 812 may be a molded portion of the mount portion 810 of the handle 802. The finger grip 812 may be configured to wrap around a finger (e.g., the index finger) of a user to provide stability during use. A lip 814 may be a curved projection of the handle 802. Cutout 816 may be curved portion of the handle 802 surrounding a cutout. The lip 814 and the cutout 816 may form an opening for the finger grip 812.

A user may grasp the ergonomic syringe attachment 800 with their hand, with their palm pointed down towards the handle 802 and their thumb on the side of the finger grip 812. In some examples, the user may wrap their hand/palm around the handle 802 and place their index finger into the finger grip pressing their index finger against the finger grip for improved stability and control of the ergonomic syringe attachment 800. The user's other fingers (e.g., non-index fingers) may curve around the handle 802 in one direction and the thumb of the user may wrap around the handle 802 in the opposite direction forming a fist around the handle 802. In other examples, the user's other fingers (e.g., non-index fingers) may curve around the neck 804 of the ergonomic syringe attachment 100 forming a pistol-like grip around the neck 804. The user's thumb may curve along a curved edge 818 of the ergonomic syringe attachment 800, where the neck 804 meets the handle 802.

Portions of the finger grip 812 may be configured to act as a vial opener. The lip 814 of the finger grip 812 may be configured to be inserted between the cap and the flange or neck of the vial. A portion of the cutout 816 may act as a fulcrum for prying the cap off a vial. Acting as a vial opener may allow a user to replace a standalone bottle opener they would otherwise carry.

In one example, a user may grasp the ergonomic syringe attachment 800 with their (e.g., non-dominant) hand, with their palm pointed down towards the handle 802 with the finger grip 812 pointed away from the user/the user's hand. Grasping the vial with their other (e.g. dominant) hand, the user may insert the lip 814 of the finger grip 812 between the cap and the flange or neck of the vial. The cap may be pried against a portion of the cutout 816 acting as a fulcrum to remove the cap from the vial.

The ergonomic syringe attachment 800 may have a U-shaped design. In this design, the neck 804 is on one side (rather than in the center/central portion) of the handle 802 and base 806. The width of the neck 804 may taper from the larger width of the handle 802 to a smaller width of base 806.

The neck 804 of the ergonomic syringe attachment 800 may have side walls 820 with an open/exposed (e.g., not enclosed) interior. A middle rib 822 may run the length of the neck 804 from the handle 802 to the base 806. The middle rib 822 may be configured to provide additional strength/support to the neck 804. The side walls 820 may continue into the base 806. The width of the base 806 may taper as it meets the neck 804. In other words, the width of the base 806 may be greater in portions further from the neck 804 than in portions closer to the neck 804.

The base 806 forks into two claws 824 at the end of the base 806 opposite the neck 804. The claws 824 form a central channel 826 in the base 806. The central channel 826 may be capsule-shaped. The two claws 824 may be configured to hold a shaft of a plunger of a syringe between the two claws 824, within the central channel 826.

Interior portions of the claws 824 may not have raised sidewalls (e.g., around the central channel 826). The height of the side walls 820 of the base 806 may become shorter/shallower (are less raised off the base 806) toward the ends of the claws 824 (away from the neck 804). In some examples, the side walls 820 taper to the floor 828 of the base 806 at the end of the claws 824.

The side walls 820 of the base 806 may have a groove throughout at least a portion of the side wall 820. The groove may be located towards a floor 828 of the base 806. The groove may be wide enough to accommodate the height of the thumb rest of a syringe. The side walls 820 may be configured to engage and couple to a thumb rest of the syringe at the groove while the shaft fits through the central channel 826. In some examples, as the width of the base 806 is narrows toward the neck 804 the distance between the groove on each side wall 820 decreases. This may allow the ergonomic syringe attachment 800 to accommodate various sized thumb rests/syringes as larger thumb rests will couple closer to the opening of the central channel 826 and smaller thumb rests (e.g., with a smaller radius/diameter) will couple closer to the neck 804 of the ergonomic syringe attachment 800.

During operation, a user may grasp the ergonomic syringe attachment 800 with one hand at the handle 802/neck 804 and engage their finder with the finger grip 812. Using their other hand, the user may slide the thumb rest of the syringe into the groove and the shaft of the syringe fits within the central channel 826. The user may place the ergonomic syringe attachment 800 with the syringe attached over the area to be depressed (e.g., attached tubing, a patient, etc.) and depress the syringe by pressing the ergonomic syringe attachment 800 on the area. The top portion of the groove (e.g. toward the handle 802) may exert force onto the thumb rest of the syringe depressing the syringe and releasing fluid in the barrel of the syringe.

It will be apparent to those skilled in the art that various modifications and variations can be made in the disclosed embodiments of the disclosed device and associated methods without departing from the spirit or scope of the disclosure. Thus, it is intended that the present disclosure covers the modifications and variations of the embodiments disclosed above provided that the modifications and variations come within the scope of any claims and their equivalents.

What is claimed is:

1. A syringe attachment device, comprising:
a base plate comprising claw-like projections at a first side, the claw-like projections forming a V-shaped channel;
a handle comprising a plurality of handle projections; and
a body comprising:
a handle mount comprising a plurality of handle bosses coupled to the plurality of handle projections;
a base plate mount coupled to the base plate with a plurality of countersink screws, the base plate mount comprising sidewalls, the sidewalls forming an opening in the base plate mount at the first side the base plate inset in the sidewalls of the base plate mount forming a flush face; and
a neck coupling the handle mount and the base plate, where the neck is coupled to the handle mount in an uncentered position along a handle mount length.

2. The syringe attachment device of claim 1, where:
the syringe attachment device is removably couplable to a syringe,
the base plate mount and the base plate configured to removably couple to a thumb rest on a shaft of a plunger of the syringe, and
the shaft of the plunger of the syringe configured to couple to the syringe attachment device between the claw-like projections of the base plate.

3. The syringe attachment device of claim 1, where:
the handle is characterized by a first length and a first width,
the handle mount is characterized by the first length and the first width,
the base plate mount comprising the first width and a second length, the second length shorter than the first length, and
the neck characterized by the first width.

4. The syringe attachment device of claim 1, where the base plate comprises a cutout with a lip and a fulcrum forming a vial opener.

5. A syringe attachment device, comprising:
a handle;
a base;
a neck component; and
a base plate coupled to the base, the base plate comprising two projections forming a channel between the two projections.

6. The syringe attachment device of claim 5, where the two projections form a V-shaped channel.

7. The syringe attachment device of claim 5, where the base comprises sidewalls, the sidewalls forming an opening at an end of the base.

8. The syringe attachment device of claim 7, where the base plate sits within the sidewalls of the base.

9. The syringe attachment device of claim 8, where an exterior face of the base plate sits flush with the sidewalls of the base.

10. The syringe attachment device of claim 7, where the sidewalls extend from an upper portion of the base.

11. The syringe attachment device of claim 10, further comprising a boss coupled to the upper portion of the base, the base plate coupled to the boss.

12. The syringe attachment device of claim 11, where a gap is formed between the upper portion of the base and the base plate at the opening.

13. The syringe attachment device of claim 12, where:
the base is configured to accept a thumb rest of a syringe at the opening, and
the syringe attachment device is couplable to the thumb rest of the syringe at the base and the base plate.

14. The syringe attachment device of claim 13, where the two projections of the base plate are couplable to a plunger shaft of the syringe.

15. A syringe attachment device, comprising:
a body comprising a handle mount, a base plate mount, and a neck coupling the handle mount and the base plate mount;
a handle coupled to the handle mount; and
a base plate coupled to the base plate mount, the base plate comprising two claws.

16. The syringe attachment device of claim 15, where the base plate comprises a vial opener cutout.

17. The syringe attachment device of claim 15, where:
the handle mount is characterized by a first width,
the base plate mount is characterized by a second width,
the neck is characterized by a third width, and
the first width, the second width, and the third width are a same width.

18. The syringe attachment device of claim 15, where:
the handle mount is characterized by a first length,
the base plate mount is characterized by a second length, and
the first length being longer than the second length.

19. The syringe attachment device of claim 18, where a first end of the handle mount aligns with a second end of the base plate mount, and a third end of the handle mount does not align with a fourth end of the base plate mount.

20. The syringe attachment device of claim 19, where the base plate mount comprises side walls forming an opening at the fourth end of the base plate mount.
